(19) United States
(12) Patent Application Publication
(10) Pub. No.: US 2020/0178843 A1
(43) Pub. Date: Jun. 11, 2020

(54) METHOD AND SYSTEM TO ACQUIRE OSCILLOMETRY MEASUREMENTS

(71) Applicant: THORASYS THORACIC MEDICAL SYSTEMS INC., Montreal (CA)

(72) Inventors: Thomas F. SCHUESSLER, Montreal (CA); Guy DRAPEAU, Montreal (CA); Geoffrey N. MAKSYM, Dartmouth (CA)

(21) Appl. No.: 16/634,321

(22) PCT Filed: Jul. 26, 2018
(86) PCT No.: PCT/CA2018/050908

§ 371 Date: Jan. 27, 2020

Related U.S. Application Data
(60) Provisional application No. 62/537,228, filed on Jul. 26, 2017.

(51) Int. Cl. A61B 5/085; A61B 5/08; A61B 5/087; A61B 5/091; A61B 5/00

(52) U.S. Cl. CPC A61B 5/085; A61B 5/0803; A61B 5/7225; A61B 5/091; A61B 5/087

(57) ABSTRACT

A method for acquiring oscillometry measurements with an oscillometry measuring system comprises receiving oscillometry measurements, using at least one processor of the oscillometry measuring system, the oscillometry measurements being from at least one oscillometry recording. Parameters are identified in the oscillometry measurements. An objective function(s) is calculated from the parameters of the oscillometry measurements. The objective function(s) is evaluated as a function of at least one predetermined threshold. The oscillometry measurements are accepted or rejected from the evaluating. Oscillometry data is output using the oscillometry measurements if accepted from the evaluating. A system for acquiring oscillometry measurements is also provided.

### CROSS-REFERENCE TO RELATED APPLICATIONS

The present application claims priority on U.S. Patent Application No. 62/537,228 filed Jul. 26, 2017, the entire contents of which are incorporated herein by reference.

### TECHNICAL FIELD

The present application relates to oscillometry measurements, for instance in the context of clinical pulmonary function testing.

### BACKGROUND OF THE ART

In clinical pulmonary function testing, it is common practice to obtain at least three repetitions of a given measurement. For lung function assessment by oscillometry (also known as the Forced Oscillation Technique, FOT), it is commonly known to acquire a minimum of three independent, valid measurements, such that the final result is calculated as the average of such three measurements. To avoid outlier measurements, the coefficient of variation (CV) between the three measurements included in the average should be less than a set threshold or else such tests are discarded.

Current oscillometry systems operated with such a procedure may therefore require a human operator to (i) assess a reading of variability such as a CV; (ii) take a decision about whether the measurements obtained are sufficiently reproducible or if further measurements need to be acquired; and (iii) select which measurements to include in or exclude from the average if additional measurements have been acquired. As a result, different criteria may be applied depending on operator skill and preferences. For example, operators have the choice to examine CVs for respiratory system resistance (R), reactance (X) or impedance (Z), each at a single frequency only or across the entire range of frequencies measured.

Using conventional techniques, additional variability may be introduced by the fact that each measurement may be subjected to varying degrees of individual quality control and breath segmentation before it is combined with other measurements to calculate average and CV. Considering a scenario in which three individual measurements of equal duration are collected, it is possible that the breath boundaries are positioned differently within the measurement such that one measurement contains one less complete breath than another. If, in addition, that measurement contains an artefact that leads to the removal of another breath, it is conceivable that the number of complete breaths contained in the three measurements contained in a given set differs by two or more breaths. Accordingly, these measurements contain different amounts of valid data, so that they no longer become comparable items for averaging. In such a scenario it would make sense to either isolate comparable episodes such as breaths, and to average and calculate CVs across such episodes rather than across measurements, or to control the system such that the likelihood of acquiring an equal number of breaths is increased, or both.

It would therefore be desirable to standardize and automate the acquisition of oscillometry measurements in order to facilitate test execution and reduce operator dependence of outcomes.

### SUMMARY

Therefore, it is an aim of the present disclosure to provide a method to acquire oscillometry measurements that addresses issues associated with the prior art.

It is a further aim of the present disclosure to provide a system to acquire oscillometry measurements that addresses issues associated with the prior art.

In accordance with an embodiment of the present disclosure, there is provided a method for acquiring oscillometry measurements with an oscillometry measuring system comprising: receiving oscillometry measurements, using at least one processor of the oscillometry measuring system, the oscillometry measurements being from at least one oscillometry recording; identifying, using the at least one processor of the oscillometry measuring system, parameters in the oscillometry measurements; calculating, using the at least one processor of the oscillometry measuring system, at least one objective function from the parameters of the oscillometry measurements, evaluating, using the at least one processor of the oscillometry measuring system, the at least one objective function as a function of at least one predetermined threshold; accepting or rejecting, using the at least one processor of the oscillometry measuring system, the oscillometry measurements from the evaluating; and outputting, using the at least one processor of the oscillometry measuring system, oscillometry data using the oscillometry measurements if accepted from the evaluating.

In the method as described herein, receiving oscillometry measurements may further include receiving oscillometry measurements delimited by breathing episodes.

In the method as described herein, receiving oscillometry measurements delimited by breathing episodes may include isolating the breathing episodes from the at least one oscillometry recording.

In the method as described herein, isolating the breathing episodes may include identifying a marker in the at least one oscillometry recording including at least one of maxima, minima, zero crossings, and crossings of pre-defined threshold values of flow or volume signals.

In the method as described herein, receiving oscillometry measurements delimited by breathing episodes may include monitoring a breath of a subject and triggering a recording of the oscillometry measurements upon detection of a desired point in a breathing cycle.

In the method as described herein, triggering a recording of the oscillometry measurements may include identifying a marker in the monitoring including at least one of maxima, minima, zero crossings, and crossings of pre-defined threshold values of flow or volume signals.

In the method as described herein, receiving oscillometry measurements may include recording the oscillometry measurements during the at least one oscillometry recording using an oscillometry measurement device from the at least one oscillometry measurement system.

In the method as described herein, identifying the parameters in the oscillometry measurements may include identifying at least one of respiratory system resistance, reactance or impedance.

In the method as described herein, calculating at least one objective function may include calculating a coefficient of variation (CV) of the resistance at a single frequency f* according to ζ=CV(R(f*)).

In the method as described herein, calculating at least one objective function may include calculating a coefficient of variation (CV) of impedance at a single frequency f* according to ζ=CV(|Z(f*)|).

In the method as described herein, calculating at least one objective function may include calculating a maximum coefficient of variation (CV) of a resistance over a range of frequencies according to ζ=max(CV(R(f))).

In the method as described herein, calculating at least one objective function may include calculating a maximum coefficient of variation (CV) of an impedance over a range of frequencies according to ζ=max(CV(|Z(f)|)).

In the method as described herein, calculating at least one objective function may include calculating an average of coefficients of variation (CV) of an impedance over Nf frequencies measured according to

$$ζ = \frac{1}{{N}_{f}} {\sum }_{i = 1}^{{N}_{f}} CV ( \vert {Z}_{fi} \vert ) .$$

In the method as described herein, calculating at least one objective function may include calculating said average of coefficients of variation (CV) of the impedance over Nf frequencies by adding a weighing function W according to

$$ζ = \frac{{\sum }_{i = 1}^{{N}_{f}} {W}_{i} \cdot CV ( \vert {Z}_{fi} \vert )}{{\sum }_{i = 1}^{{N}_{f}} {W}_{i}} .$$

In the method as described herein, calculating at least one objective function may include calculating said average of coefficients of variation (CV) of the impedance over Nf frequencies by calculating a root mean squared value according to

$$ζ = {( \frac{{\sum }_{i = 1}^{{N}_{f}} {W}_{i} \cdot {CV ( \vert {Z}_{fi} \vert )}^{2}}{{\sum }_{i = 1}^{{N}_{f}} {W}_{i}} )}^{\frac{1}{2}} .$$

In the method as described herein, calculating at least one objective function may include calculating said average of coefficients of variation (CV) of the impedance over Nf frequencies by calculating a root mean squared value in which an order of the root marches a power p to which each CV is elevated to according to

$$ζ = {( \frac{{\sum }_{i = 1}^{{N}_{f}} {W}_{i} \cdot {CV ( \vert {Z}_{fi} \vert )}^{p}}{{\sum }_{i = 1}^{{N}_{f}} {W}_{i}} )}^{\frac{1}{p}} .$$

In the method as described herein, calculating at least one objective function may include calculating a sum of squared standard deviations divided by a sum of squared means of |Z| across the Nf frequencies measured according to

$$ζ = \frac{{\sum }_{i = 1}^{{N}_{f}} {SD ( \vert {Z}_{fi} \vert )}^{2}}{{\sum }_{i = 1}^{{N}_{f}} {\overline{{Z}_{fi}}}^{2}} .$$

In the method as described herein, calculating at least one objective function may include calculating a sum of squared standard deviations divided by a sum of squared means of |Z| across the Nf frequencies measured, with an added weighing function W according to

$$ζ = \frac{{\sum }_{i = 1}^{{N}_{f}} {W}_{i} \cdot {SD ( \vert {Z}_{fi} \vert )}^{2}}{{\sum }_{i = 1}^{{N}_{f}} {W}_{i} \cdot {\overline{{Z}_{fi}}}^{2}} .$$

In the method as described herein, calculating at least one objective function may include calculating a sum of standard deviations elevated to a power p divided by a sum of means of |Z| elevated to the power p, across Nf frequencies measured, including a weighing function W according to

$$ζ = \frac{{\sum }_{i = 1}^{{N}_{f}} {W}_{i} \cdot {SD ( \vert {Z}_{fi} \vert )}^{p}}{{\sum }_{i = 1}^{{N}_{f}} {W}_{i} \cdot {\overline{{Z}_{fi}}}^{p}} .$$

In the method as described herein, calculating at least one objective function may include determining if a minimum number Nₘᵢₙ of oscillometry measurements is reached prior to calculating the at least one objective function.

The method as described herein may further comprise combining oscillometry measurements if the minimum number Nₘᵢₙ of oscillometry measurements is exceeded, the combining include all permutations having at least a minimum number Nₐᵥ of oscillometry measurements required for averaging.

In the method as described herein, evaluating the at least one objective function may include evaluating the at least one objective function using one of the permutations selected as a function of the at least one predetermined threshold.

In the method as described herein, calculating at least one objective function may include calculating the objective function using an average of the parameters for the oscillometry measurements.

In the method as described herein, accepting or rejecting the oscillometry measurements may include rejecting the oscillometry measurements if a maximum number Nₘₐₓ of oscillometry measurements is exceeded.

In the method as described herein, evaluating the oscillometry measurements may include grading the oscillometry measurements as a function of the at least one predetermined threshold.

In accordance with another embodiment of the present disclosure, there is also provided a system for acquiring oscillometry measurements comprising: a processing unit; and a non-transitory computer-readable memory communicatively coupled to the processing unit and comprising computer-readable program instructions executable by the processing unit for: receiving oscillometry measurements, using at least one processor of the oscillometry measuring system, the oscillometry measurements being from at least one oscillometry recording; identifying, using the at least one processor of the oscillometry measuring system, parameters in the oscillometry measurements; calculating, using the at least one processor of the oscillometry measuring system, at least one objective function from the parameters of the oscillometry measurements, evaluating, using the at least one processor of the oscillometry measuring system, the at least one objective function as a function of at least one predetermined threshold; accepting or rejecting, using the at least one processor of the oscillometry measuring system, the oscillometry measurements from the evaluating; and outputting, using the at least one processor of the oscillometry measuring system, oscillometry data using the oscillometry measurements if accepted from the evaluating.

In the system as described herein, receiving oscillometry measurements may include receiving oscillometry measurements delimited by breathing episodes.

In the system as described herein, receiving oscillometry measurements delimited by breathing episodes may include isolating the breathing episodes from the at least one oscillometry recording.

In the system as described herein, isolating the breathing episodes may include identifying a marker in the at least one oscillometry recording including at least one of maxima, minima, zero crossings, crossings of pre-defined threshold values of flow or volume signals.

In the system as described herein, receiving oscillometry measurements delimited by breathing episodes may include monitoring a breath of a subject and triggering a recording of the oscillometry measurements upon detection of a desired point in a breathing cycle.

In the system as described herein, triggering a recording of the oscillometry measurements may include identifying a marker in the monitoring including at least one of maxima, minima, zero crossings, crossings of pre-defined threshold values of flow or volume signals.

In the system as described herein, receiving oscillometry measurements may include recording the oscillometry measurements during the at least one oscillometry recording using an oscillometry measurement device from the at least one oscillometry measurement system.

In the system as described herein, identifying the parameters in the oscillometry measurements may include identifying at least one of respiratory system resistance, reactance or impedance.

In the system as described herein, calculating at least one objective function may include calculating a coefficient of variation (CV) of the resistance at a single frequency f* according to ζ=CV(R(f*)).

In the system as described herein, calculating at least one objective function may include calculating a coefficient of variation (CV) of impedance at a single frequency f* according to ζ=CV(|Z(f*)|).

In the system as described herein, calculating at least one objective function may include calculating a maximum coefficient of variation (CV) of a resistance over a range of frequencies according to ζ=max(CV(R(f))).

In the system as described herein, calculating at least one objective function may include calculating a maximum coefficient of variation (CV) of an impedance over a range of frequencies according to ζ=max(CV(|Z(f)|)).

In the system as described herein, calculating at least one objective function may include calculating an average of coefficients of variation (CV) of an impedance over N_{f} frequencies measured according to

$$ζ = \frac{1}{{N}_{f}} {\sum }_{i = 1}^{{N}_{f}} CV ( \vert {Z}_{fi} \vert ) .$$

In the system as described herein, calculating at least one objective function may include calculating said average of coefficients of variation (CV) of the impedance over N_{f} frequencies by adding a weighing function W according to

$$ζ = \frac{{\sum }_{i = 1}^{{N}_{f}} {W}_{i} \cdot CV ( \vert {Z}_{fi} \vert )}{{\sum }_{i = 1}^{{N}_{f}} {W}_{i}} .$$

In the system as described herein, calculating at least one objective function may include calculating said average of coefficients of variation (CV) of the impedance over N_{f} frequencies by calculating a root mean squared value according to

$$ζ = {( \frac{{\sum }_{i = 1}^{{N}_{f}} {W}_{i} \cdot {CV ( \vert {Z}_{fi} \vert )}^{2}}{{\sum }_{i = 1}^{{N}_{f}} {W}_{i}} )}^{\frac{1}{2}} .$$

In the system as described herein, calculating at least one objective function may include calculating said average of coefficients of variation (CV) of the impedance over N_{f} frequencies by calculating a root mean squared value in which an order of the root marches a power p to which each CV is elevated to according to

$$ζ = {( \frac{{\sum }_{i = 1}^{{N}_{f}} {W}_{i} \cdot {CV ( \vert {Z}_{fi} \vert )}^{p}}{{\sum }_{i = 1}^{{N}_{f}} {W}_{i}} )}^{\frac{1}{p}} .$$

In the system as described herein, calculating at least one objective function may include calculating a sum of squared standard deviations divided by a sum of squared means of |Z| across the N_{f} frequencies measured according to

$$ζ = \frac{{\sum }_{i = 1}^{{N}_{f}} {SD ( \vert {Z}_{fi} \vert )}^{2}}{{\sum }_{i = 1}^{{N}_{f}} {\overline{{Z}_{fi}}}^{2}} .$$

In the system as described herein, calculating at least one objective function may include calculating a sum of squared standard deviations divided by a sum of squared means of |Z| across the N_{f} frequencies measured, with an added weighing function W according to

$$ζ = \frac{{\sum }_{i = 1}^{{N}_{f}} {W}_{i} \cdot {SD ( \vert {Z}_{fi} \vert )}^{p}}{{\sum }_{i = 1}^{{N}_{f}} {W}_{i} \cdot {\overline{{Z}_{fi}}}^{p}} .$$

In the system as described herein, calculating at least one objective function may include calculating a sum of standard deviations elevated to a power p divided by a sum of means of |Z| elevated to the power p, across Nf frequencies measured, including a weighing function W according to

$$ζ = \frac{{\sum }_{i = 1}^{{N}_{f}} {W}_{i} \cdot {SD ( \vert {Z}_{fi} \vert )}^{p}}{{\sum }_{i = 1}^{{N}_{f}} {W}_{i} \cdot {\overline{{Z}_{fi}}}^{p}} .$$

In the system as described herein, calculating at least one objective function may include determining if a minimum number Nₘᵢₙ of oscillometry measurements is reached prior to calculating the at least one objective function.

The system as described herein may further comprise combining oscillometry measurements if the minimum number Nₘᵢₙ of oscillometry measurements is exceeded, the combining include all permutations having at least a minimum number Nₐᵥ of oscillometry measurements required for averaging.

In the system as described herein, evaluating the at least one objective function may include evaluating the at least one objective function using one of the permutations selected as a function of the at least one predetermined threshold.

In the system as described herein, calculating at least one objective function may include calculating the objective function using an average of the parameters for the oscillometry measurements.

In the system as described herein, accepting or rejecting the oscillometry measurements may include rejecting the oscillometry measurements if a maximum number Nₘₐₓ of oscillometry measurements is exceeded.

In the system as described herein, evaluating the oscillometry measurements may include grading the oscillometry measurements as a function of the at least one predetermined threshold.

The system as described herein may further comprise the oscillometry device for recording the at least one oscillometry recording.

In accordance with a further embodiment of the present disclosure, there is also provided a system for acquiring oscillometry measurements comprising: an acquisition module configured for receiving repeated oscillometry measurements from a device and identifying parameters from the oscillometry measurements; and an objective function evaluator module calculating at least one objective function from the parameters of the repeated oscillometry measurements, and evaluating the at least one objective function as a function of at least one predetermined threshold, the objective function evaluator module accepting or rejecting oscillometry measurements from the evaluating; whereby the system outputs oscillometry data using accepted oscillometry measurements from the evaluating.

### DESCRIPTION OF THE DRAWINGS

FIG. 1 is a block diagram of a system to acquire oscillometry measurements in accordance with the present disclosure;

FIG. 2 is a flowchart of a method to assess acquired oscillometry measurements in accordance with the present disclosure;

FIG. 3 is a flowchart of an extended method to assess acquired oscillometry measurements in accordance with the present disclosure, with grading;

FIG. 4 is a flowchart of an extended method to assess episodes such as breaths isolated from acquired oscillometry measurements in accordance with the present disclosure;

FIG. 5 is a flowchart of an extended method to assess acquired oscillometry measurements in accordance with the present disclosure, wherein measurement acquisition is controlled such that the onset of all measurements coincides with the same point in the breathing cycle and/or occurs if conditions to confirm stability of the breathing pattern have been met;

FIG. 6 shows exemplary average coefficients of variation from variable patients using no qualification, manual qualification by an expert and automatic qualification as per a method of the present disclosure;

FIG. 7 shows exemplary individual coefficients of variation from variable patients using no qualification, manual qualification by an expert and automatic qualification as per a method of the present disclosure; and

FIG. 8 is a schematic view of an exemplary oscillometry measurement device that may be used with the methods and systems described herein.

### DETAILED DESCRIPTION

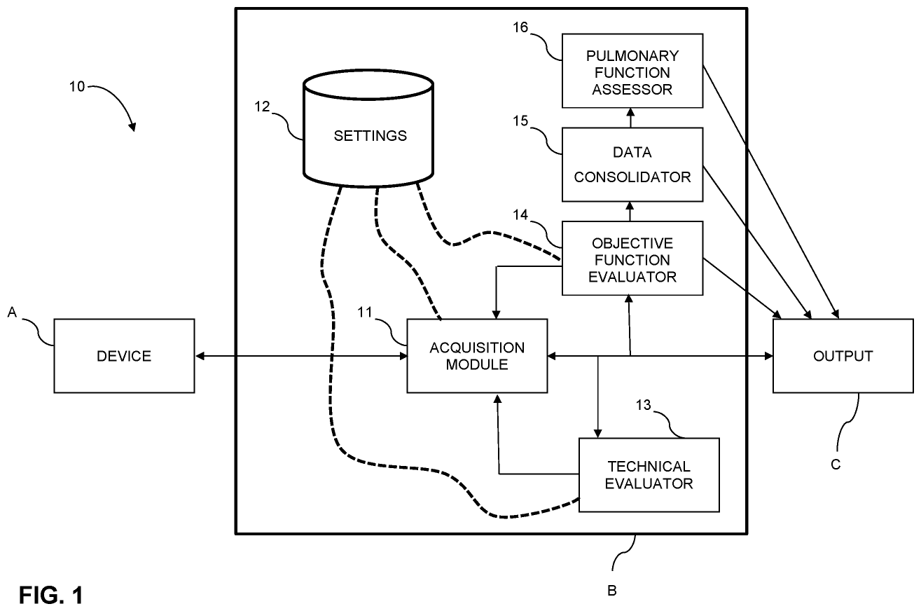

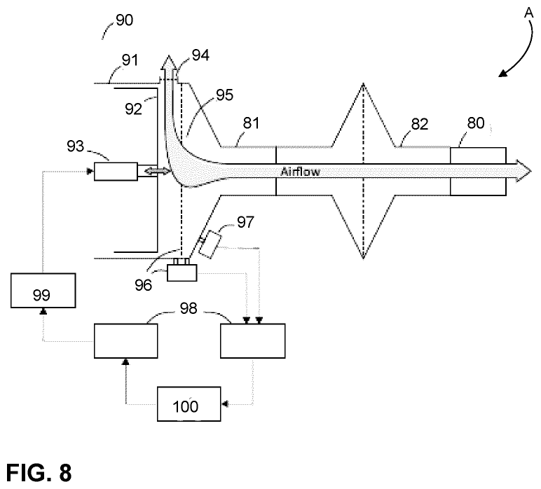

Referring to the drawings, and more particularly to FIG. 1, there is illustrated a system 10 to acquire oscillometry measurements in accordance with the present disclosure, in such a way that the acquired oscillometry measurements comply with desired acquisition parameters. The system 10 may then use the oscillometry measurements to assess the pulmonary mechanics of a patient. The system 10 is illustrated as having an oscillometry measurement device A that typically consists of at least a breathing pathway with a patient port and an atmosphere port, an oscillator adding an oscillatory component to the patient's breathing, and a flow meter measuring airflow in and out of the patient. For example, the oscillator may be a loudspeaker, a mechanical ventilator capable of producing oscillatory flows, a vibrating mesh in the flow pathway or a piezoelectric beam bending actuated devices among possible oscillators. The flow meter may be a conventional screen a pneumotachograph, an ultrasonic flow meter, a wave tube or a variable orifice flow meter, to name a few options among others. The device A may be known as an oscillometry apparatus or system, an airwave oscillometry apparatus or system, an impulse oscillometry apparatus or system, a forced-oscillation technique (FOT) apparatus or system, and/or a forced oscillometry apparatus or system, among possible names. A non-limitative embodiment of an oscillometry measurement device that may be used with the methods and systems of the present disclosure is described hereinafter with reference to FIG. 8. The device A is used in conjunction with an oscillometry acquisition processor B that produces an output C in any appropriate format. For example, the output C may be a monitor, a data file, a parameter, a set of parameters, a signal, a table, a graph, a chart or a report, each providing data quantifying the oscillometry measurements, or an average, standard deviation, median, maximum, minimum or similar consolidated measure thereof, and in some instances assessing pulmonary mechanics, or used subsequently to assess or assist in assessing pulmonary mechanics. Although the device A, the processor B and the output C are shown in FIG. 1 as being discrete components, the processor B and output C may be integrated to the device A. Processor B and output C may also be connected in any appropriate way to the device A, and may for instance be embodied as a computer, a tablet, etc. The processor B may be part of one or more computers, and may include multiple processor units as well. However, for simplicity, reference is made herein to a processor B. The processor B must have sufficient computing speed to receive and interpret data produced by the device A in situ, for example in real-time or quasi-real time. Moreover, the processor B, as described below, must perform tasks with the acquired oscillometry measurements to determine if it meets testing requirements. In these circumstances, the processor B must perform the tasks in situ, in the ongoing clinical session, and often between oscillometry measurements, as a rejected measurement may influence the testing protocol. Accordingly, not only are objective functions and grading schemes calculated by the processor B, as described herein, but the determined objective functions and grading schemes are used to adjust the workflow and prompt users to collect additional recordings if—and only if—they are needed. Thus, the processor B performs, during an ongoing clinical testing session, the determination as to whether or not each acquired oscillometry measurement meets the predetermined testing requirements or not, and can advise the user accordingly—for example after each acquired measurement—and then automatically adjust the workflow accordingly. Due to the fact that the testing protocol may include the evaluation of multiple objective functions using signal interpretation from the device A, and real time analysis as to the suitability of each newly acquirement measurement relative to the determined testing requirements, appropriate computing capacity is required for the processor B.

The processor B has an acquisition module 11 that is programmed to perform oscillometry measurement testing. The acquisition module 11 activates and controls the device A, receives raw data signals from the device A, and may convert them to an initial set of useful oscillometry measurement data, identifying and/or calculating measurement parameters that may include respiratory system resistance, reactance or impedance, to name a few options. The acquisition module 11 may also segment the measurements into periodic episodes such as breaths, a measurement being constituted typically of one or more complete episodes. To clarify, the system 10 and methods described herein performing patient testing, by which one or more oscillometry recordings are performed. An oscillometry recording may be generally defined as the moment extending from the start to the end of one contiguous session of recording sensor data for the patient using the device A. Accordingly, an oscillometry recording may include one or more breathing cycles (i.e., breaths), a breath also referred to as an episode. Episodes may also be defined by segments of a breath, such as the inspiratory phase and the expiratory phase of a breath, or singular points or regions of a breath, such as end-inspiration or end-expiration. For the purposes of the present disclosure, an oscillometry recording may include one or more oscillometry measurements, as the oscillometry measurement is an episode or a full recording, or segments of a full recording, that contains the necessary data to calculate a cost function representative of pulmonary function. Accordingly, the expression “oscillometry measurement” is used herein to include one or more episodes of breathing, a full oscillometry recording, and/or segments of a full oscillometry recording. The expression “breathing episodes” is intended to include full breaths, segments of breaths, and/or recordings of same.

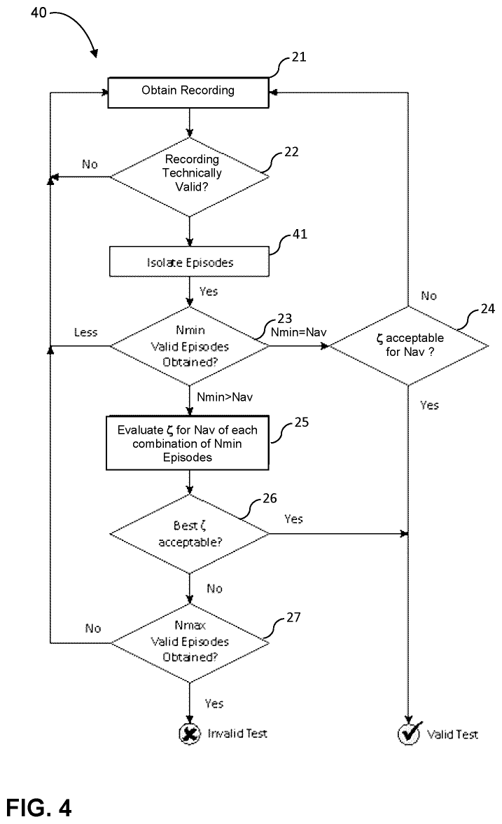

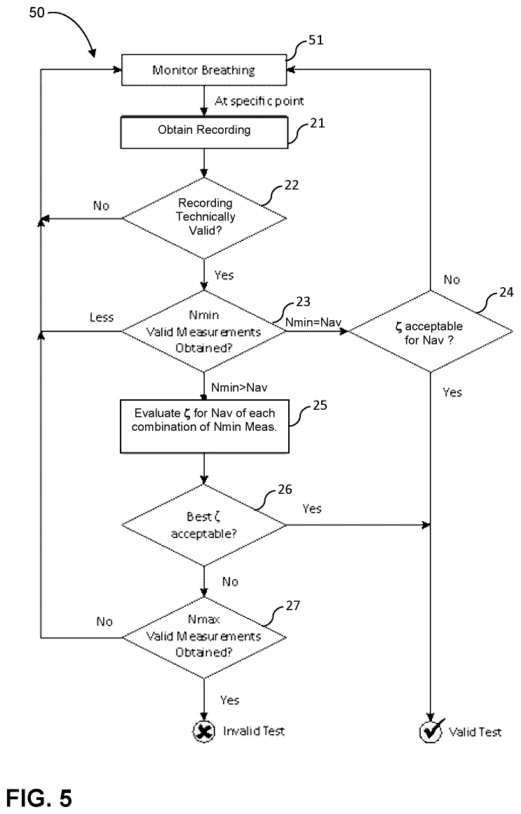

The acquisition module 11 may have the capacity to acquire live streams of breathing pressure and/or flow data immediately prior to an oscillometry recording, and may have the capacity to segment such pre-recording data streams to isolate episodes of breathing, in order to control the device A or delimit measurements such that the onset of a recording (i) always coincides with the same point in the breathing cycle, and/or (ii) occurs only if conditions to confirm stability of the breathing pattern have been met. The steps performed by the acquisition module 11 are described in further detail below, with reference to FIGS. 4 and 5 for example. The acquisition module 11 may also drive the testing via the output C by displaying information on how the testing should be conducted. For this purpose, the acquisition module 11 may have access to a settings database 12, shown as being an integral part of the processor B, but alternatively accessible remotely. In fact, various modules of the processor B may be cloud-based, and accessible via telecommunications, as one possible embodiment in addition to one in which the modules are integrated in the processor B. The settings database 12 may also include settings controlling the position of the onset of a measurement within the breathing cycle, the segmentation of a measurement into episodes such as breaths, the evaluation of objective function(s) (ζ), acceptance criteria ζ, for and other settings such as the number of oscillometry measurements to be averaged (Nₐᵥ), the minimum number of oscillometry measurements or episodes required (Nₘᵢₙ) and the maximum number of oscillometry measurements or episodes permitted (Nₘₐₓ), all of which may be related to ensuring the oscillometry testing is properly and repeatably conducted and meets standards for being used to properly assess pulmonary function. The settings are described in further details hereinafter.

The acquisition module 11 may also operate with a technical evaluator module 13 that is configured to evaluate whether the individual oscillometry measurements or episodes are technically valid. For example, factors that may influence the technical validity include duration, clipping of raw data, insufficient magnitude of breathing or oscillatory pressure, flow or volume waveforms, breathing irregularities, insufficient number of breaths, poor mathematical indicators such as coherence or signal-to-noise ratio, artefacts such as leaks, coughs or swallowing detected, and insufficient remaining data volume after exclusion of such artefacts. Thus, during the monitoring phase 51 (see FIG. 5), the technical evaluator module 13 is operable to confirm the stability of the breathing pattern, for example by determining whether a set number of repeatable breaths (e.g. three consecutive breaths with comparable inspiratory time, expiratory time, tidal volume, peak flow, and/or flow shape index, etc.) before triggering a measurement capture. The technical evaluator module 13 monitors these factors and may indicate if any given oscillometry measurement or episode is, by itself, technically valid. Based on this result, the acquisition module 11 may count the number of oscillometry measurements or episodes taken, as the count value of number of measurements or episodes is used in determining if the testing is valid or has failed, as discussed hereinafter. In an embodiment, the system 10 is without the technical evaluator module 13.

An objective function evaluator module 14 receives the data of valid oscillometry measurements or episodes, which may include raw data as well as any output produced by the acquisition module 11, and performs the function of determining if a combination of multiple oscillometry measurements or episodes constitutes a valid test. Accordingly, the objective function evaluator module 14 is programmed or accesses the objective functions that are identified as being relevant to this determination, and has the algorithms necessary to calculate the objection function values from the oscillometry data. The objective functions are also detailed hereinafter. The objective function evaluator module 14 may evaluate one or more of the objective functions, and may grade the test based on the objective function(s).

If the objective function evaluator module 14 determines that the oscillometry recordings and/or oscillometry measurements meet applicable test standards and thus constitute a valid test, selected oscillometry measurements are output via the output C. The selected oscillometry measurements may be used for calculations to an appropriate value, such as an average, standard deviation, median, maximum, minimum or similar consolidated measure, by the data consolidation module 15. Moreover, using a pulmonary function assessment module 16, the processor B may also perform further analysis of the consolidated measures provided by data consolidation module 15 in order to further assist in or facilitate the analysis and interpretation of the data and the assessment of pulmonary function. Accordingly, the output C may display or output the data from any one of the acquisition module 11, the objective function evaluator module 14, data consolidation module 15, and/or the pulmonary function assessment module 16.

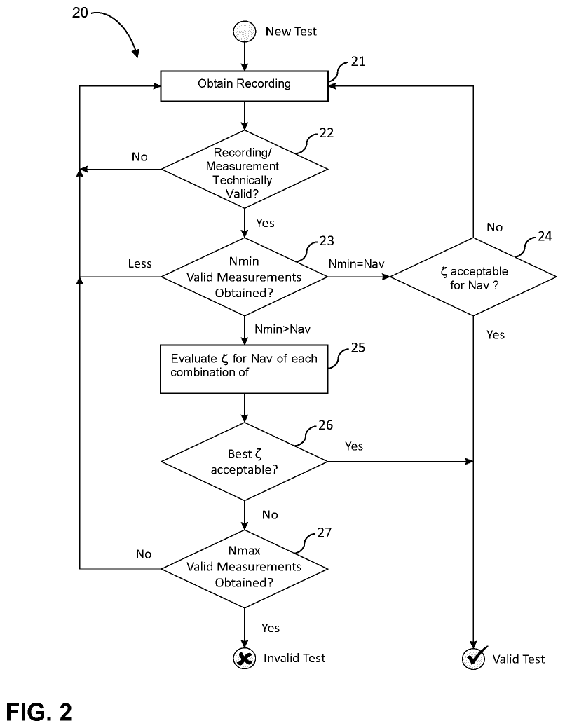

The system 10 may operate using a method to acquire oscillometry measurements, as shown at 20 in FIG. 2. According to an embodiment, the method 20 requires:
at least one objective function (ζ) that captures variability between repeated oscillometry measurements, although a plurality of objective functions may be used in the method 20;
an acceptance criterion for ζ, related to oscillometry measurements;
a minimum number of oscillometry measurements to be averaged (Nₐᵥ);
a minimum number of oscillometry measurements required (Nₘᵢₙ); and
a maximum number of oscillometry measurements permitted (Nₘₐₓ).

Without loss of generality, it may be assumed that Nₘₐₓ is greater than or equal to Nₘᵢₙ, Nₘᵢₙ is greater than or equal to Nₐᵥ, Nₘₐₓ is greater than Nₐᵥ, and Nₐᵥ is greater than zero. Then, the method 20 can be described as having at least some of the following steps:

At 21, upon initiation of a new test, a first oscillometry recording is taken or obtained. The recording employs an appropriate device for oscillometry measurements, as detailed in the system 10.

At 22, the oscillometry recording and/or oscillometry measurement thereof are individually validated for technical validity according to established criteria. Measurements and/or recordings that are not technically valid do not count towards Nₘᵢₙ and Nₘₐₓ.

At 23, if Nₘᵢₙ valid measurements have not been obtained, an additional measurement is initiated, and 21, 22 and 23 may be repeated until Nₘᵢₙ valid measurements are acquired. If at least Nₘᵢₙ and exactly Nₐᵥ valid oscillometry measurements have been obtained, ζ is(are) first evaluated for the Nₐᵥ valid oscillometry measurements at 24. If ζ meets its acceptance criterion as determined at 24 for Nₐᵥ, the test is valid and complete, i.e., the acquisition of oscillometry measurements respects the standards of the test and a pulmonary function assessment may be performed based on the measurements and/or the system confirms it has successfully performed a valid oscillometry test.

According to an embodiment, the average Nₐᵥ used in the calculation of the cost function(s) ζ may be smaller to Nₘᵢₙ, as mentioned above. As an example, Nₐᵥ is set to 3 and Nₘᵢₙ=5. In such an example, the system 10 may take for example five measurements, resulting in possibly six permutations of three measurements (if averaging is limited to exactly Nₐᵥ measurements), or eleven permutations of three to five measurements (if averaging is allowed for any at least Nₐᵥ measurements). The average of Nₐᵥ=3 may therefore be optimized for as many as eleven possible combinations. Consequently, the results of the test may be calculated as the average of the Nₐᵥ measurements. If ζ does not meet its acceptance criterion as determined at 24, another measurement is obtained at 21.

The method 20 may include obtaining multiple oscillometry measurements in 21 before 22 and 23, such that the number of valid measurements exceeds Nₘᵢₙ. For example, if the measurements are episodes of an oscillometry recording, the system 10 and method 20 may have numerous episodes that exceed Nₘᵢₙ upon obtaining the sensed data of the first oscillometry recording.

When more than Nₘᵢₙ valid measurements have been obtained, ζ in one embodiment is evaluated for each combination of at least Nₐᵥ measurements contained within the >Nₐᵥ available measurements, as per 25. The combinations must therefore include the averaging of at least Nₐᵥ valid measurements, but may also exceed the minimum number of Nₐᵥ valid measurements. For example, if the minimum number of Nₐᵥ valid measurements required in the average is three, and there are four valid measurements obtained (N1, N2, N3 and N4), the combinations may be as follows: (N1, N2, and N3), (N1, N3 and N4), (N2, N3 and N4), (N1, N2, N3 and N4). If ζ for at least one combination of at least Nₐᵥ valid measurements has a value that meets the acceptance criterion, the test is valid and complete, and results are calculated as the average of the at least Nₐᵥ measurements of the combination whose ζ value passes the acceptance criterion by the biggest margin. In another embodiment, ζ is evaluated for each combination of exactly Nₐᵥ measurements contained within the >Nₐᵥ available measurements at 25. In the above example, only combinations (N1, N2, and N3), (N1, N3 and N4) and (N2, N3 and N4) are evaluated; if ζ for at least one combination of exactly Nₐᵥ valid measurements has a value that meets the acceptance criterion, the test is valid and complete, and results are calculated as the average of the exactly Nₐᵥ measurements of the combination whose ζ value passes the acceptance criterion by the biggest margin.

Accordingly, step 25 may entail numerous permutations through the numerous combinations, with each permutation involving the calculation of the cost function ζ. As the system 10 and method 20 must be done in situ to be operable, the processor(s) involved in computing the data for the system 10 and method 20 must have suitable processing speed in spite of the complexity of and volume of data computed in step 25.

As per 26, if ζ for not one combination of at least or exactly Nₐᵥ valid measurements meets the acceptance criterion, an additional recording(s) is(are) initiated, if less than the Nₘₐₓ measurements have been obtained as determined at 27. Still as per 27, if Nₘₐₓ measurements have been obtained and none of the ζ for any of combination of at least/exactly Nₐᵥ measurements contained within the Nₘₐₓ available measurements meets the acceptance criterion as determined in 25, the test is invalid and has failed. The oscillometry test may not be used as they do not meet test standards.

The method 20 described above can function with a variety of different objective functions measured with the system 10, in the context of oscillometry measurements for subsequent pulmonary function assessment. An enumeration of objective functions is provided below, and the system 10 and method 20 of the present disclosure may use one or more of the cost functions, individually, or in any appropriate combination of two or more of these cost functions. These may include:
The coefficient of variation (CV) of the resistance at a single frequency f* that is considered representative or most important (e.g., at 5 Hz), i.e.

ζ=CV(R(f*)).
The CV of the magnitude of impedance at a single frequency f* that is considered representative or most important, i.e.

ζ=CV(|Z(f*)|).
The maximal CV of the resistance over a range of frequencies, i.e.

ζ=max(CV(R(f)))
The maximal CV of the magnitude of impedance over a range of frequencies, i.e.

ζ=max(CV(|Z(f)|))
An average over the values of CV(|Z|) at the N_{f} frequencies measured, i.e.

$$ζ = \frac{1}{{N}_{f}} {\sum }_{i = 1}^{{N}_{f}} CV ( \vert {Z}_{fi} \vert )$$

Said average over the values of CV(|Z|) at the N_{f} frequencies measured, in which an added weighing function W may be used to permit attributing more importance to specific frequencies or correcting for an unevenly distributed frequency spectrum, i.e.

$$ζ = \frac{{\sum }_{i = 1}^{{N}_{f}} {W}_{i} \cdot CV ( \vert {Z}_{fi} \vert )}{{\sum }_{i = 1}^{{N}_{f}} {W}_{i}}$$

The Root Mean Squared (RMS) value of CV(|Z|) across the N_{f} frequencies measured, again including an optional weighing function W, i.e.

$$ζ = {( \frac{{\sum }_{i = 1}^{{N}_{f}} {W}_{i} \cdot {CV ( \vert {Z}_{fi} \vert )}^{2}}{{\sum }_{i = 1}^{{N}_{f}} {W}_{i}} )}^{\frac{1}{2}}$$

A higher powered equivalent of an RMS value of CV(|Z|) across the N_{f} frequencies measured, wherein the order of the root matches the power p that each CV is elevated to, again including an optional weighing function W, i.e.

$$ζ = {( \frac{{\sum }_{i = 1}^{{N}_{f}} {W}_{i} \cdot {CV ( \vert {Z}_{fi} \vert )}^{p}}{{\sum }_{i = 1}^{{N}_{f}} {W}_{i}} )}^{\frac{1}{p}}$$

The sum of the squared standard deviations divided by the sum of the squared means of |Z| across the N_{f} frequencies measured, i.e.

$$ζ = \frac{{\sum }_{i = 1}^{{N}_{f}} {SD ( \vert {Z}_{fi} \vert )}^{2}}{{\sum }_{i = 1}^{{N}_{f}} {\overline{\vert {Z}_{fi} \vert}}^{2}}$$

Said sum of the squared standard deviations divided by the sum of the squared means of |Z| across the N_{f} frequencies measured, with an added weighing function W, i.e.

$$ζ = \frac{{\sum }_{i = 1}^{{N}_{f}} {W}_{i} \cdot {SD ( \vert {Z}_{fi} \vert )}^{2}}{{\sum }_{i = 1}^{{N}_{f}} {\overline{\vert {Z}_{fi} \vert}}^{2}}$$

The sum of the standard deviations elevated to a power p divided by the sum of the means of |Z| elevated to the same power p, across the N_{f} frequencies measured, again including an optional weighing function W, i.e.

$$ζ = \frac{{\sum }_{i = 1}^{{N}_{f}} {W}_{i} \cdot {SD ( \vert {Z}_{fi} \vert )}^{p}}{{\sum }_{i = 1}^{{N}_{f}} {\overline{\vert {Z}_{fi} \vert}}^{p}}$$

It should be noted that using objective functions for the CV values listed as the first four items above (with no particular order of importance), the inclusion or exclusion of a test is based on the variability at a single frequency, therefore these objective functions fail to capture the overall variability of spectral oscillometry measurements. In contrast, averaging functions as per the items after the first four items take into consideration all frequencies. The combination of a weighing factor in the average over the values of CV(|Z|) at the N_{f} frequencies measured, and the squaring or further elevation of the summands provide mechanisms to further control the relative contributions of individual frequencies, as well as the sensitivity of the method 20 towards increased variability at only a small number of frequencies within the spectrum. Again, processing speed must support such calculations for the system 10 and method 20 to be operable in situ.

A commonly used acceptance criterion for inclusion or rejection of a test in the context of oscillometry measurements requires the objective function not to exceed a predefined threshold value, i.e., ζ≤ζₘₐₓ.

In a further embodiment, the method 20 could be extended to use a combination of a multitude of objective functions ζ related to oscillometry measurements, wherein independent acceptance criteria are applied for each objective function ζ and connected via logical operations. For example, the two objective functions

$$ζ = {( \frac{{\sum }_{i = 1}^{{N}_{f}} {W}_{i} \cdot {CV ( \vert {Z}_{fi} \vert )}^{p}}{{\sum }_{i = 1}^{{N}_{f}} {W}_{i}} )}^{\frac{1}{p}}$$

$$and$$

$$θ = max ( CV ( \vert Z ( f ) \vert ) )$$

could be meaningfully combined so that tests are accepted when ζ≥ζₘₐₓ and Θ≤Θₘₐₓ.

In another embodiment, the method 20 may employ quality grading to determine if the oscillometry measurements are suitable for a proper assessment of pulmonary function. The method 20 may employ quality grading by introducing multiple thresholds ζᵢ, so that ζ₁<ζ₂< . . . <ζₘₐₓ, wherein passing the most stringent threshold ζ₁ results in the best possible rating. For the example of a total of three thresholds (including ζₘₐₓ), this would result in the following grading:

| Value of ζ | Rating |
|---|---|
| ≤ζ₁ | “A” - Excellent |
| >ζ₁ but ≤ζ₂ | “B” - Good |
| >ζ₂ but ≤ζₘₐₓ | “C” - Fair |
| >ζₘₐₓ | “X” - Rejected |

In another embodiment, thresholds on ζ are combined with other factors, such as the number of measurements required to reach a certain grading. For the example of Nₘᵢₙ=3, Nₘₐₓ=5 and a total of three thresholds (including ζₘₐₓ), this would result in the following grading:

| | | Number of Measurements Needed | | |
|---|---|---|---|---|
| | | 3 | 4 | 5 |
| Value of ζ | ≤ζ₁ | “A” - Excellent | “A” - Excellent | “B” - Good |
| | >ζ₁ but ≤ζ₂ | “B” - Good | “B” - Good | “C” - Fair |
| | >ζ₂ but ≤ζₘₐₓ | “C” - Fair | “C” - Fair | “C” - Fair |
| | >ζₘₐₓ | “X” - Rejected | “X” - Rejected | “X” - Rejected |

In another embodiment where multiple objective functions are used, multiple thresholds per objective functions can be combined to produce a grading. For the example of a total of three thresholds for two objective functions ζ and Θ, this would result in the following grading:

| | | Value of Θ | | | |
|---|---|---|---|---|---|
| | | ≤Θ₁ | >Θ₁ but Θ₂ | >Θ₂ but ≤Θₘₐₓ | >Θₘₐₓ |
| Value of ζ | ≤ζ₁ | “A” - Excellent | “A” - Excellent | “B” - Good | “X” - Rejected |
| | >ζ₁ but ≤ζ₂ | “A” - Excellent | “B” - Good | “C” - Fair | “X” - Rejected |
| | >ζ₂ but ≤ζₘₐₓ | “B” - Good | “C” - Fair | “X” - Rejected | “X” - Rejected |
| | >ζₘₐₓ | “X” - Rejected | “X” - Rejected | “X” - Rejected | “X” - Rejected |

In another embodiment, the grading scheme is calculated as a decimal score S that assumes its maximal value Sₘₐₓ when variability is zero and approaches zero as ζ approaches ζₘₐₓ, i.e.

$$S = \{ \begin{matrix}{S}_{max} \cdot \frac{{ζ}_{max} - ζ}{{ζ}_{max}} for ζ \leq {ζ}_{max} \\ 0 for ζ > {ζ}_{max}\end{matrix}$$

Accordingly, the methods of the present disclosure may provide, in addition or as an alternative to a binary pass/fail value, a grading of the oscillometry measurements. In particular, the methods may include the use of one or more objective functions, to determine if the oscillometry measurements are suitable for a proper assessment of pulmonary function. Likewise, the system 10 of the present disclosure has the capacity of performing a self-assessment of oscillometry measurements performed thereon—with suitable processing speed —, to assist an operator in the decision making regarding the quality of the pulmonary function tests on a subject.

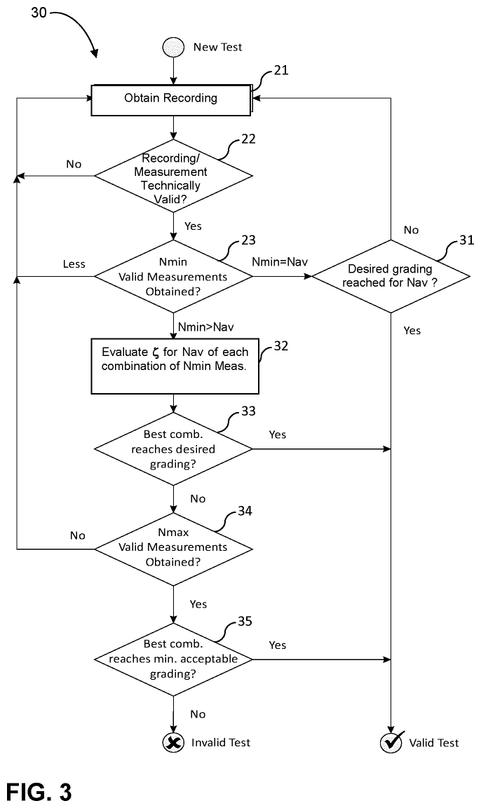

In the presence of a grading scheme as described above, the method 20 described above and shown in FIG. 2 may be extended to include two thresholds, in the manner shown as 30 in FIG. 3, still in the context of oscillometry measurements. As methods 20 and 30 share some steps, like reference numerals will indicate similar steps. The two thresholds of method 30 may be:
a more stringent “desired grading” that the method aims to reach, and
a less stringent “minimum acceptable grading” that must be reached for a test to be considered valid.

With reference to FIG. 3, a patient test according to the method 30 may proceed as follows. In similar fashion to method 20, at 21, upon initiation of a new test, a first oscillometry recording and/or measurement is taken. At 22, the measurement is individually validated for technical validity according to established criteria. Measurements that are not technically valid do not count towards Nₘᵢₙ and Nₘₐₓ. At 23, when Nₘᵢₙ valid measurements have been obtained in a condition of Nₐᵥ=Nₘᵢₙ, ζ is(are) first evaluated and graded at 31, such that a preliminary grading is established. Still in 30, if the preliminary grading reaches or exceeds the desired grading upon comparison with the threshold(s), the test is valid and complete, and results are calculated as the average of the Nₐᵥ measurements. The oscillometry measurements are suitable for a proper assessment of pulmonary function, and the acquisition by the method 30 is completed, for the assessment of pulmonary mechanics to be performed based on the oscillometry measurements. If does not meet the desired grading as determined at 30, another measurement is obtained at 21.

The method 30 may include obtaining multiple measurements in 21 before 22 and 23, such that the number of valid measurements exceeds Nₘᵢₙ. As another possibility, Nₐᵥ<Nₘᵢₙ as detailed above in steps 23 and 25. When more than Nₘᵢₙ valid measurements and/or more than Nₐᵥ valid measurements have been obtained, the objective function in 32 is evaluated and a preliminary grading is attributed to the average of each combination of at least Nₐᵥ measurements (or exactly Nₐᵥ measurements) contained within the available measurements, as detailed above for 25. In 33, if the grading for Nₐᵥ of at least one combination meets at least the desired grading, the test is valid and complete, and results are calculated as the average of the Nₐᵥ measurements of the combination having at least Nₐᵥ measurements (or exactly Nₐᵥ measurements) with the highest grading (if the grading is quantitative), or that passes the desired grading threshold by the biggest margin (if the grading is binned, e.g. for a letter grading).

In 34, if the average of not one combination of at least Nₐᵥ valid measurements meets the desired grading, one or more additional recordings are initiated at 21, if less than Nₘₐₓ measurements have been obtained, as per 34. The additional measurement(s) may serve to improve the grading of the oscillometry measurement. Still in 34, if Nₘₐₓ measurements have been obtained and the average of none of the combinations of at least Nₐᵥ measurements (or exactly Nₐᵥ measurements) contained within the various Nₘₐₓ available measurements reaches the desired grading, the method 30 reaches 35. According to 35, if at least one grading reaches or exceeds a minimum acceptable grading, the test is valid and complete, and results are calculated as the average of those at least Nₐᵥ measurements (or exactly Nₐᵥ measurements) with the highest grading (if the grading is quantitative), or that passes the minimum acceptable grading threshold by the biggest margin (if the grading is binned, e.g. for a letter grading). Still at 35, if the minimum acceptable grading threshold is not reached by any combination of measurements, the test is invalid and has failed.

As described above, the methods 20 and 30 may apply to oscillometry recordings as a whole, or to isolated episodes. In the latter case, with reference to FIG. 4, the system 10 may or may not apply a method 40 for isolating some episodes within an oscillometry recording, to perform the assessment on isolated episodes such as breaths rather than entire oscillometry recordings. As methods 20, 30 and 40 share some steps, like reference numerals will indicate similar steps. More specifically, the method 20 described above and shown in FIG. 2 may be extended to include the additional step 41 of isolating such episodes. The isolating of episodes may for example be performed by a review of the data of oscillometry recordings to identify markers delimiting an episode. For example, the markers may include maxima, minima, zero crossings or crossings of pre-defined threshold values of the flow or volume signals, to name a few of many possible options. Processing speed must support such isolation for the system 10 and method 40 to be operable in situ. Although not shown, the step 41 could be added to the method 30 of FIG. 3 as well, with the step 41 occurring for example between steps 21 and 22.

As described above, the device A may be controlled in such a way that the onset of the oscillometry recording (i) always coincides with the same point in the breathing cycle, and/or (ii) occurs only if conditions to confirm stability of the breathing pattern have been met. Therefore, the method 20 described above and shown in FIG. 2 may be extended into method 50 of FIG. 5. The method 50 includes the additional step 51 of monitoring the breathing pattern until the desired point in the breathing cycle is reached. The step 51 may trigger the step 21 of obtaining the oscillometry recording, for instance synchronized with a predetermined episode, by monitoring any one of the markers identified above for 41, or by predictive timing based on previous episodes. For example, if the system determines that a given patient has a stable breath duration of six seconds, a recording can be triggered five seconds into a breath to be certain that a new breath starts early in the recording. Processing speed must support such monitoring and triggering for the system 10 and method 50 to be operable in situ. Although not shown, the step 51 could be added to the method 30 of FIG. 3 as well, with the step 51 occurring before step 21.

The methods 20, 30, 40 and 50 of FIGS. 2, 3, 4 and 5, respectively, may be integrated into the processor B of the system 10. The processor B or processors B of the system 10 may include a non-transitory computer-readable memory communicatively coupled to the processing unit(s) B and comprising computer-readable program instructions executable by the processing unit for executing at least some of the steps of methods 20, 30, 40 and 50. The various steps of the methods 20, 30, 40 and 50 may be executed by different modules of the system 10. At the outset, the system 10 therefore aims to output oscillometry measurements that comply with an established testing protocol and standards in situ. This allows corrective measures and a suitable number of oscillometry measurements to be taken during a clinical session, to avoid having to retest a patient at a later time.

In the methods 20, 30, 40 and 50 described above, the concepts of minimum number Nₘᵢₙ of oscillometry measurements and average Nₐᵥ of a number of oscillometry measurements are applied. In all of these methods, the average Nₐᵥ used in the calculation of the cost function(s) may be smaller or equal to Nₘᵢₙ, in addition to having the capacity of being higher as well. As an example, Nₐᵥ is set to n=3 and Nₘᵢₙ=5, for any of methods 20, 30, 40 and 50. In such an example, the system 10 may take for example five measurements, resulting in possibly six permutations of three measurements (if averaging is limited to exactly Nₐᵥ measurements), or eleven permutations of three to five measurements (if averaging is allowed for any at least Nₐᵥ measurements). The average of Nₐᵥ=3 may therefore be optimized for as many as eleven possible combinations.

As another possibility, Nₐᵥ and Nₘᵢₙ are equal to one another, as explored with steps 24 (FIG. 2) and 31 (FIG. 3). In the example, they are both equal to three, but the system 10 may take more than three measurements in a >Nₘᵢₙ scenario (e.g., five measurements), whereby Nₐᵥ may be optimized when selecting among the eleven combinations. Consequently, in such an embodiment, some degree of optimization is possible if more than Nₘᵢₙ measurement are taken.

Exemplary Testing Data

For illustrative purposes only, to illustrate the system 10 and some aspects of the methods described herein, exemplary testing data is provided below. As the testing data was obtained in particular settings, it should only be viewed as a particular non-limitative embodiment.

To test method 20, from a larger dataset, 40 high-variability patients (including a variety of pathologies) were extracted, for whom tests containing at least four oscillometry measurements had been recorded. First, a calculation was performed for both the CV of impedance magnitude at a measurement frequency of 5 Hz, CV(Z₅), and the Root Mean Squared value of CV(|Z|) across all measured frequencies, CVᵣₘₛ(Z), as described above (and, in case of the CVᵣₘₛ(Z), with all weights equalling 1.0). CV(Z₅) and CVᵣₘₛ(Z) were each evaluated both across all measurements collected, representing the unqualified tests results as might be produced by an unskilled or novice user.

Next, each test was examined by an expert physician and researcher with ample experience in reviewing oscillometry data, with no time constraint as such review would far exceed any in situ assessment and would not be possible in the conditions of operation of the system 10 and methods 20, 30, 40 and 50. For 35 of the patient tests, the expert manually selected three valid measurements, and CV(Z₅) and CVᵣₘₛ(Z) were each evaluated across the selected measurements, representing the qualified test results produced by an expert user.

Finally, the method 20 was applied to 34 out of the 35 patient tests selected by the expert, using Nₐᵥ=3, Nₘᵢₙ=5 and Nₘₐₓ=5. One test was excluded from further analysis because it contained only 4 measurements and therefore did not comply with Nₘᵢₙ=5. CV(Z₅) and CVᵣₘₛ(Z) were each evaluated across the measurements selected by method 20, representing the automatically qualified test results produced by our method.

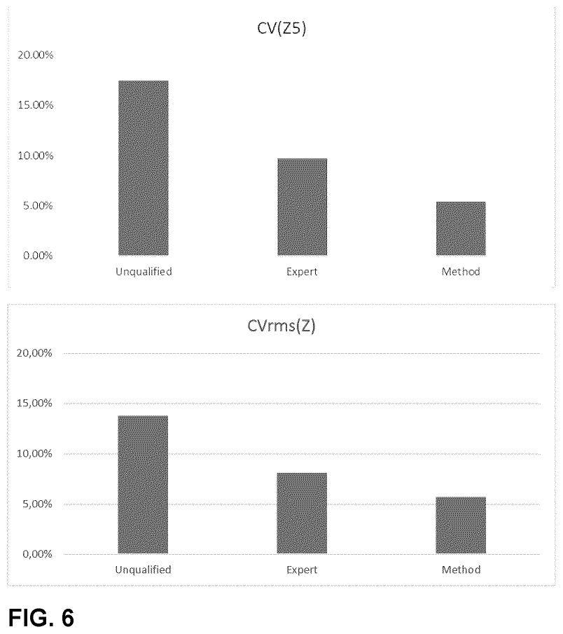

FIG. 6 shows the average CVs for all three cases. A substantial and significant (paired t-test; all p<0.00001) difference can be observed between the unqualified and the expert-qualified test results for both CV(Z₅) and CVᵣₘₛ(Z) that underscores the importance of a good qualification method on the reproducibility of outcomes and illustrates the dependence of the prior art on operator skill, in non in situ settings as it is not possible to perform such human analysis in the conditions set out above for the system 10 and methods 20, 30, 40 and 50.

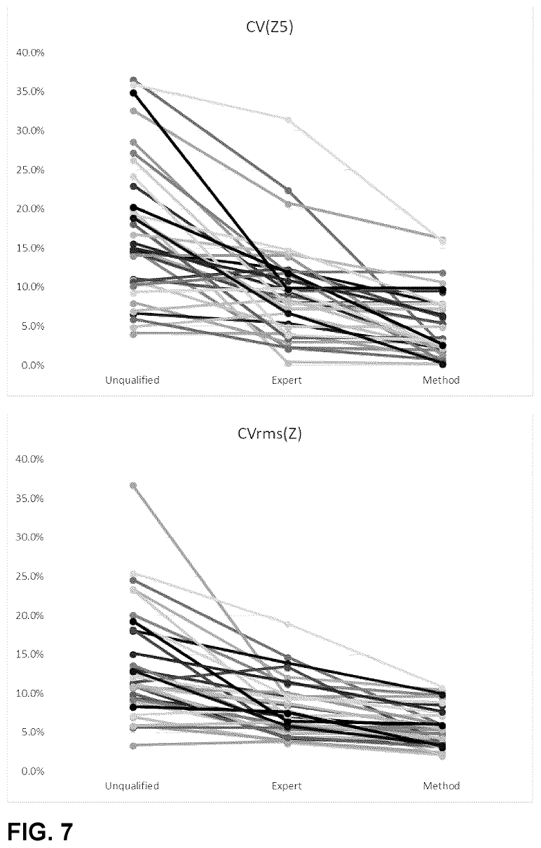

FIG. 6 also shows that the automated qualification using method 20 produced coefficients of variation that were lower (all p<0.00001) than both the corresponding expert-qualified values and the unqualified values, for both CV(Z₅) and CVᵣₘₛ(Z), in addition to being performable in situ, e.g., in real-time or quasi-real time. On average, as an example only, the method 20 improved CV(Z₅) and CVᵣₘₛ(Z) by 12.1% and 8.1%, respectively, whereas the expert improved CV(Z₅) and CVᵣₘₛ(Z) by a lesser 7.8% and 5.7%, respectively. As illustrated in FIG. 7, method 20 improved both CV(Z₅) and CVᵣₘₛ(Z) for every one of the 34 patient tests, whereas the expert failed to improve or even worsened CV(Z₅) and CVᵣₘₛ(Z) in 5 and 4 cases, respectively.

To establish exclusion thresholds, statistics were performed on the expert-qualified data to identify the values that correspond to the 95^{th} percentile, which for CV(Z₅) and CVᵣₘₛ(Z) equalled 15.2% and 15.8%, respectively. Applying the threshold for CV(Z₅) to the unqualified, expert-qualified and method 20—qualified datasets resulted in 17, 3 and 2 excluded tests, respectively, whereas applying the threshold for CVᵣₘₛ(Z) to the three datasets yielded 10, 1 and zero excluded tests, respectively.

To further test the method 30 in the same exemplary dataset, thresholds were also established corresponding to the 75^{th} and 85^{th} percentile of the expert-qualified data, so that patient test could be graded as follows:

| Expert-qualified percentile | CV(Z₅) | CVᵣₘₛ(Z) | Grading |
|---|---|---|---|
| 75% | <11.2% | <9.7% | A |
| 85% | <12.6% | <11.7% | B |
| 95% | <15.2% | <15.8% | C |
| | ≥15.2% | ≥15.8% | X |

Applying the thresholds for CV(Z₅), the grading was as follows:

| Grading | Unqualified | Expert | Method 30 |
|---|---|---|---|
| A | 11 | 23 | 31 |
| B | 0 | 5 | 1 |
| C | 6 | 3 | 0 |
| X | 17 | 3 | 2 |

Applying the thresholds for CVᵣₘₛ(Z), the grading was as follows:

| Grading | Unqualified | Expert | Method 30 |
|---|---|---|---|
| A | 10 | 25 | 30 |
| B | 8 | 4 | 4 |
| C | 6 | 4 | 0 |
| X | 10 | 1 | 0 |

The data presented above illustrate that methods 20, 30, 40 and 50 offer means for automatic in-situ quality control and test workflow management that cannot be perform by a human operator due to the demand of analytical evaluations and decisions.

Exemplary Oscillometry Measurement Device A

The oscillometry measurement device A of FIG. 1 may be embodied for instance in the manner shown in FIG. 8. However, this is merely an option among others for obtaining oscillometry measurements with the system 10 or methods 20, 30, 40 and 50 of the present disclosure. Other exemplary devices are described above.

With the oscillometry measurement device A, a subject breathes through the device A via a mouthpiece 80 connected to a duct 81 via a bacterial filter 82. The other side of duct 81 is connected to an oscillator 90 that may include an oscillator housing 91, an oscillator piston 92, a linear actuator 93 capable of oscillating piston 92, and an atmosphere port 94 with a defined impedance to atmosphere. The interior of the oscillator 90 contains a front chamber 95 that communicates with the airway opening and is subject to the pressure swings generated by the oscillator 90 and the subject's breathing. The front chamber 95 further contains a sensor 96 for measuring flow and a sensor 97 for measuring pressure. Analog/digital converters and digital/analog converters 98 interface sensors 96 and 97 as well as a power amplifier 99 driving the actuator 44 to a processor 100. The processor 100 may or may not be part of the processor B. In an embodiment, the processor 100 is a microprocessor integrated to the device A such that the device A may be separate from the processor B, with the processor B receiving signals from the processor 100.

1.-25. (canceled)

26. A system for acquiring oscillometry measurements comprising:
a processing unit; and
a non-transitory computer-readable memory communicatively coupled to the processing unit and comprising computer-readable program instructions executable by the processing unit for:
receiving oscillometry measurements, using at least one processor of the oscillometry measuring system, the oscillometry measurements being from at least one oscillometry recording;
identifying, using the at least one processor of the oscillometry measuring system, parameters in the oscillometry measurements;
calculating, using the at least one processor of the oscillometry measuring system, at least one objective function from the parameters of the oscillometry measurements,
evaluating, using the at least one processor of the oscillometry measuring system, the at least one objective function as a function of at least one predetermined threshold;
accepting or rejecting, using the at least one processor of the oscillometry measuring system, the oscillometry measurements from the evaluating; and
outputting, using the at least one processor of the oscillometry measuring system, oscillometry data using the oscillometry measurements if accepted from the evaluating.

27. The system according to claim 26, wherein receiving oscillometry measurements includes receiving oscillometry measurements delimited by breathing episodes.

28. The system according to claim 27, wherein receiving oscillometry measurements delimited by breathing episodes includes isolating the breathing episodes from the at least one oscillometry recording.

29. (canceled)

30. The system according to claim 27, wherein receiving oscillometry measurements delimited by breathing episodes includes monitoring a breath of a subject and triggering a recording of the oscillometry measurements upon detection of a desired point in a breathing cycle.

31. (canceled)

32. The system according to claim 26, wherein receiving oscillometry measurements includes recording the oscillometry measurements during the at least one oscillometry recording using an oscillometry measurement device from the at least one oscillometry measurement system.

33. The system according to claim 26, wherein identifying the parameters in the oscillometry measurements includes identifying at least one of respiratory system resistance, reactance or impedance.

34. The system according to claim 26, wherein calculating at least one objective function includes calculating a coefficient of variation (CV) of the resistance at a single frequency f* according to ζ=CV(R(f*)).

35. The system according to claim 26, wherein calculating at least one objective function includes calculating a coefficient of variation (CV) of impedance at a single frequency f* according to ζ=CV(|Z(f*)|).

36. The system according to claim 26, wherein calculating at least one objective function includes calculating a maximum coefficient of variation (CV) of a resistance over a range of frequencies according to ζ=max(CV(R(f))).

37. The system according to claim 26, wherein calculating at least one objective function includes calculating a maximum coefficient of variation (CV) of an impedance over a range of frequencies according to ζ=max(CV(|Z(f)|)).

38. The system according to claim 26, wherein calculating at least one objective function includes calculating an average of coefficients of variation (CV) of an impedance over N_{f} frequencies measured according to
$$ζ = \frac{1}{{N}_{f}} {\sum }_{i = 1}^{{N}_{f}} CV ( \vert {Z}_{fi} \vert ) .$$

39. The system according to claim 38, wherein calculating at least one objective function includes calculating said average of coefficients of variation (CV) of the impedance over N_{f} frequencies by adding a weighing function W according to
$$ζ = \frac{{\sum }_{i = 1}^{{N}_{f}} {W}_{i} \cdot CV ( \vert {Z}_{fi} \vert )}{{\sum }_{i = 1}^{{N}_{f}} {W}_{i}} .$$

40. The system according to claim 38, wherein calculating at least one objective function includes calculating said average of coefficients of variation (CV) of the impedance over N_{f} frequencies by calculating a root mean squared value according to
$$ζ = {( \frac{{\sum }_{i = 1}^{{N}_{f}} {W}_{i} \cdot {CV ( \vert {Z}_{fi} \vert )}^{2}}{{\sum }_{i = 1}^{{N}_{f}} {W}_{i}} )}^{\frac{1}{2}} .$$

41. The system according to claim 38, wherein calculating at least one objective function includes calculating said average of coefficients of variation (CV) of the impedance over N_{f} frequencies by calculating a root mean squared value in which an order of the root marches a power p to which each CV is elevated to according to
$$ζ = {( \frac{{\sum }_{i = 1}^{{N}_{f}} {W}_{i} \cdot {CV ( \vert {Z}_{fi} \vert )}^{p}}{{\sum }_{i = 1}^{{N}_{f}} {W}_{i}} )}^{\frac{1}{p}} .$$

42. The system according to claim 26, wherein calculating at least one objective function includes calculating a sum of squared standard deviations divided by a sum of squared means of |Z| across the N_{f} frequencies measured according to
$$ζ = \frac{{\sum }_{i = 1}^{{N}_{f}} {SD ( \vert {Z}_{fi} \vert )}^{2}}{{\sum }_{i = 1}^{{N}_{f}} {\overline{\vert {Z}_{fi} \vert}}^{2}}$$

43. The system according to claim 26, wherein calculating at least one objective function includes calculating a sum of squared standard deviations divided by a sum of squared means of |Z| across the N_{f} frequencies measured, with an added weighing function W according to
$$ζ = \frac{{\sum }_{i = 1}^{{N}_{f}} {W}_{i} \cdot {SD ( \vert {Z}_{fi} \vert )}^{2}}{{\sum }_{i = 1}^{{N}_{f}} {\overline{\vert {Z}_{fi} \vert}}^{2}} .$$

44. The system according to claim 26, wherein calculating at least one objective function includes calculating a sum of standard deviations elevated to a power p divided by a sum of means of |Z| elevated to the power p, across N_{f} frequencies measured, including a weighing function W according to
$$ζ = \frac{{\sum }_{i = 1}^{{N}_{f}} {W}_{i} \cdot {SD ( \vert {Z}_{fi} \vert )}^{p}}{{\sum }_{i = 1}^{{N}_{f}} {\overline{\vert {Z}_{fi} \vert}}^{p}} .$$

45. The system according to claim 26, wherein calculating at least one objective function includes determining if a minimum number Nₘᵢₙ of oscillometry measurements is reached prior to calculating the at least one objective function.

46.-48. (canceled)

49. The system according to claim 26, wherein accepting or rejecting the oscillometry measurements includes rejecting the oscillometry measurements if a maximum number Nₘₐₓ of oscillometry measurements is exceeded.

50. The system according to claim 26, wherein evaluating the oscillometry measurements includes grading the oscillometry measurements as a function of the at least one predetermined threshold.

51.-52. (canceled)
